(19) United States
(12) Patent Application Publication
(10) Pub. No.: US 2016/0128898 A1
(43) Pub. Date: May 12, 2016

(54) DEVICE FOR A CONTROLLED HEART-LUNG RESUSCITATION IN THE EVENT OF A CARDIAC ARREST

(71) Applicant: Sebastian MÖSSMER, München (DE)

(72) Inventors: CHRISTIAN HAGL, MÜNCHEN (DE); KATHRIN ROHLK, MUNCHEN (DE)

(21) Appl. No.: 14/441,664

(22) PCT Filed: Nov. 8, 2013
(86) PCT No.: PCT/DE2013/000665

§ 371 Date: Dec. 28, 2015

(30) Foreign Application Priority Data
Nov. 8, 2012 (DE) 10 2012 021 828.1
Feb. 11, 2013 (DE) 10 2013 002 428.5
Apr. 15, 2013 (DE) 10 2013 006 469.4

(51) Int. Cl. A61H 31/00; A61N 1/39

(52) U.S. Cl. CPC A61H 31/007; A61H 31/005; A61N 1/39; A61H 2201/5071

(57) ABSTRACT

The invention relates to a device (1) for a controlled cardio-pulmonary resuscitation, said device enabling the user to carry out a quick and incomplicated resuscitation of a human body in the event of a cardiac arrest. The geometric dimensions of the device (1) according to the invention are comparatively small, ranging approximately from 10 and 25 cm in diameter and approximately 6 to 12 cm in height. During use, a mechanical pressure (F) is exerted onto a first pressure transmitting means (2, 22), said mechanical pressure generating a signal (S3, S4), which is perceptible to human organs, upon reaching a maximal exerted force (Fₘₐₓ), wherein the signal is triggered by a spring element (24, 44) which is arranged between the pressure transmitting means (2, 22) and the base plate (3, 33).

The present invention is concerned with a device for the controlled cardiopulmonary resuscitation from cardiac arrest with a clearly audible signal when reaching a threshold pressure, in particular with a device with a special pen and molding system, which facilitates the handling during application of the device.

Such devices are known in the art from the group consisting of U.S. Pat. No. 4,554,910 in the prior art. This document discloses an apparatus for cardiopulmonary resuscitation in cardiac arrest with at least one pressure transmitting means and at least one pressure receiving member and a pressure gauge that the occurrence of a mechanical limit pressure (Fmax) a perceivable by human senses signal (S) generated. Between the at least one pressure transfer means and the at least one pressure receiving element is disposed a spring system with two different springs which produces on reaching a predetermined limit pressure is through one of the two springs an audible first click signal and upon release of the limit pressure a second. The first and second acoustic click signal is generated by means of a U-shaped leaf spring which is arranged approximately in the center of the second spring and is designed as a helical spring. A disadvantage of such a device for cardiopulmonary resuscitation in cardiac arrest, it is felt that the mechanical pressure must be centrally act on the first pressure transmitting means always to achieve the desired effect for resuscitation. This is not possible in practical use.

A similar elongated device has become known from CN 201304070 Y, which also has two pressure transmitting means, wipe which a helical spring is arranged and produced upon reaching a threshold pressure an audible signal that the user signals to relieve the pressure transmitting means again. A disadvantage of this device, it is felt that it is hardly possible to obtain a stable position in the thorax in an emergency.

The WO 2006/101400 A1 document discloses an apparatus for manual pressure generation on the chest of a human body. This device has a mechanical tone generator, which generates a sound upon reaching a predetermined pressure. For this purpose, a plate is placed in a holder that holds the plate in a curved bias and produces a sound when you press the panel. The pressure measurement itself is carried out by means of another mechanism, which is described in more detail in 2004/056303 A1 WO. A disadvantage of such a device for heart-lung massage it is felt that the interaction of all mechanical components are too complicated due to the absolute reliability requirement of such a device, so that the desired security can not be guaranteed.

Furthermore, from the document DE 1491611 a portable heart massage apparatus has become known which consists of a base plate and an overhead punch, wherein the die is actuated cyclically by means of a pneumatic mechanism and acts on the thorax of the human body. Since it is generally at a cardiac arrest it arrives, perform resuscitation as soon as possible, the available devices are often in their handling too costly and complicated to operate, so that valuable time can be lost to the revival of the human body, which far-reaching consequences.

Moreover, defibrillators are well known in the prior art and are in intensive care units, in operating rooms, in emergency rooms, as well as in rescue vehicles kept and since the 1990s, increasingly, in public buildings such as railway stations, airports and other places for an application by medical laymen.

It is therefore an object of the present invention to provide a simple and easy by laymen in this field to use device for cardiopulmonary resuscitation, which is able to have one hand acting a controlled, secure printing cyclically on the chest of the human body, which is largely independent of the position of the center of pressure on the unit.

This object is solved by the characterizing features of the main claims. Further inventive features are disclosed in the dependent claims and the detailed description.

The present invention provides a device for controlled cardiopulmonary resuscitation is presented, which is able to carry out a rapid and uncomplicated reanimation of a human body in cardiac arrest. The geometrical dimensions of the device according to the invention are relatively low and are between about 10 and 25 cm in diameter and about 6 to 12 inches in height. In the application is applied to a pressure-transmitting means is a mechanical pressure (F) cyclically generating a perceptible in human organs signal when reaching a maximum exertion of force (Fmax), which is effected due to the cooperation of spring elements disposed between the first pressure transmission means and a base plate are arranged. In the housing of the device is a defibrillator is optionally integrated, which is applied immediately after the heart-lung massage.

The inventive device for controlled cardiopulmonary resuscitation of the human body in cardiac arrest with at least one pressure transfer means with a pressure gauge, which is located at least directly and/or indirectly with the treatable chest at a predetermined location and the occurrence of a limit pressure (Fmax) a by sense organs produces perceptible signal (S3, S4), is characterized by at least a two-dimensional one-piece spring element which detects when exposed to a mechanical pressure (F) an adjustable limit pressure (Fmax) and upon release of the mechanical pressure (Fmax) back to the original starting position of the spring element returns spontaneously, being both upon reaching the pressure limit (Fmax) and the spring back into the starting position produces a clearly audible signal (S) which acts both mechanically and acoustically to the spring element bearing elements. This pressure indicator device comprises at least one spring system with a plurality of spring elements which cooperate when exercising the mechanical pressure (F), wherein at least one spring element is flat and one piece and at least two spring elements are arranged laterally of the flat spring element.

A further device according to the invention for the controlled cardiopulmonary resuscitation of the human body for cardiac arrest is characterized by an electronic unit (Defibrillator), which due to a signal (S) emits an adjustable current pulse which acts on the body to be treated of the patient.

It is advantageous that a flat-storey spring element which when exposed to a mechanical pressure (F) shows an adjustable limit pressure (Fmax) and spontaneously springs back upon release of the mechanical pressure in the original starting position of the spring element, whereby both in reaching the limit value (Fmax) and the resetting to the start position sounds a clearly audible signal, which acts both mechanically and acoustically to the load-bearing spring element environment.

It is also advantageous for the device that the cross-sectional shape is trapezoidal in approximately through the center of at least one spring element, wherein G1 represents the lower baseline, and g2, the upper baseline of the trapezoid.

Another advantage is the fact that the two sides are taking S1 and S2 of the trapezoid with the base line g1 an acute angle (α1, α2)), where (α1) and (α2) are not necessarily equal.

It is also advantageous that the wall thickness (d) of the planar one-piece shell between 0.5 and 1 mm, preferably about 0.3 mm, when the material used is a spring steel.

It is furthermore advantageous, that the edges of the inner and outer periphery having deformations and notches affect and define the stiffness of the spring element.

It is also advantageous that at least one pressure transfer means is a relatively rigid element that is at least one further pressure transmitting means directly or indirectly connected.

Another advantage is the fact that at least one pressure transmitting means is connected to the body to be treated directly with the contact surface of the pressure transmitting means is adapted to the anatomical conditions.

It is also advantageous that the pressure gauge and/or pressure limit display can be formed both electronically and mechanically and/or optically.

It is furthermore advantageous that the signal (S) generating unit is equipped at least with a pressure receiving element.

A further advantage is that at least one pressure transmitting means is formed back expanding.

It is furthermore advantageous that at a predetermined location, preferably on the bottom or at the hand area on the top side a characterizing element is arranged, which determines the correct positioning of the device on the chest.

It is furthermore advantageous that the apparatus can comprise an integrated electronics with at least one sensor which receives the mechanical pressure and by the electronics, a signal (S) generated.

A further advantage is to be seen in that the electronics is powered by an internal or external power source.

It is advantageous that at least indirectly and/or directly, the inventive device is placed with the treated chest at a predetermined location.

Further, it is advantageous that the device has a switch, so that on reaching the adjustable limit pressure (Fmax) at least one electrical contact is actuated.

It is advantageous, moreover, that at the at least one two-dimensional one-piece spring element, at least one further spring element is arranged, which compensates the mechanical pressure on the at least one pressure transfer means and the pressure transmitting means is cushioned quasi floating.

Further, it is advantageous that the further spring elements are arranged around the first flat spring element and are formed in one embodiment as a spiral or helical springs.

Further, it is advantageous that the energy storage device, for example, a capacitor or battery that is charged by means of a manually actuated dynamo, actuated by the massage movements.

It is also advantageous that the apparatus includes an accumulator, a DC/DC converter and a capacitor in a circuit with a control unit, wherein the voltage by means of the DC/DC converter is produced, with the capacitor to a previously set Energy is charged. By pressing a button, the capacitor releases the stored energy, about 200 to 360 joules, to the patient. The voltage is up to 4000 volts and is between 3 and 40 ms. The current intensity achieved with conventional body resistance between 50 ohms and 100 ohms up to about 50 amps. The necessary for this capacitor has a capacitance of 45 microfarads to 500 microfarads.

This stored energy is delivered through large electrodes are what pushed either to the victim's chest with your hands (the so-called “paddles”) or are stuck on the chest (“adhesive electrodes” or “Fast-patches”). Especially in publicly accessible defibrillators (“PAD” Public Access Defibrillator) are—to simplify the operation and reduce the risk of electric shock for the user—practically only used adhesive electrodes.

The output circuit provides for the generation of specific pulse shapes. The control unit regulates the charging of the capacitor, passes the output circuit and also ensures that will be discharged in absence of shock, the capacitor via an internal resistor (protection circuit).

Modern defibrillators work biphasic. This means that from the output stage not only a shock is delivered, but that are submitted by voltage change on the paddles and power surges in the reverse direction. Biphasic devices allow thus to make do with the same effectiveness at a lower power output and lower damage to the heart muscle. Modern biphasic defibrillators measure before the energy release the body resistance (impedance) of the patient by means of glued electrodes and match amperage and voltage to this resistance. Slender, small patients with low impedance condition so less power than z. B. overweight, large patients.

The working method with this device for resuscitation of the cardiopulmonary activity is characterized by the following steps:
P1 positioning the device on the chest of the human body;
P2 exerting mechanical pressure (F) on the surface of the device until a pressure limit (Fmax) is reached; and
P3 produce a perceivable by human sense organs first signal (S3, S3′);
P4 Complete withdrawal of mechanical pressure (F) according to perception of the signal (S);
P5 repetition of the mechanical pressure (F) on the surface of the device, wherein the steps P2 to P4 are repeated.
P6 applying the electrodes to the body to be treated; and
P7 discharge of the energy store.

It is advantageous that the energy storage device can be charged by both a battery and a mechanically operated to Dynamo, wherein the electronic unit is integrated into the housing of the heart-lung massage device.

Further inventive features are in the description and the subclaims.

In the now following, the invention will be explained in more detail with reference to drawings. It shows

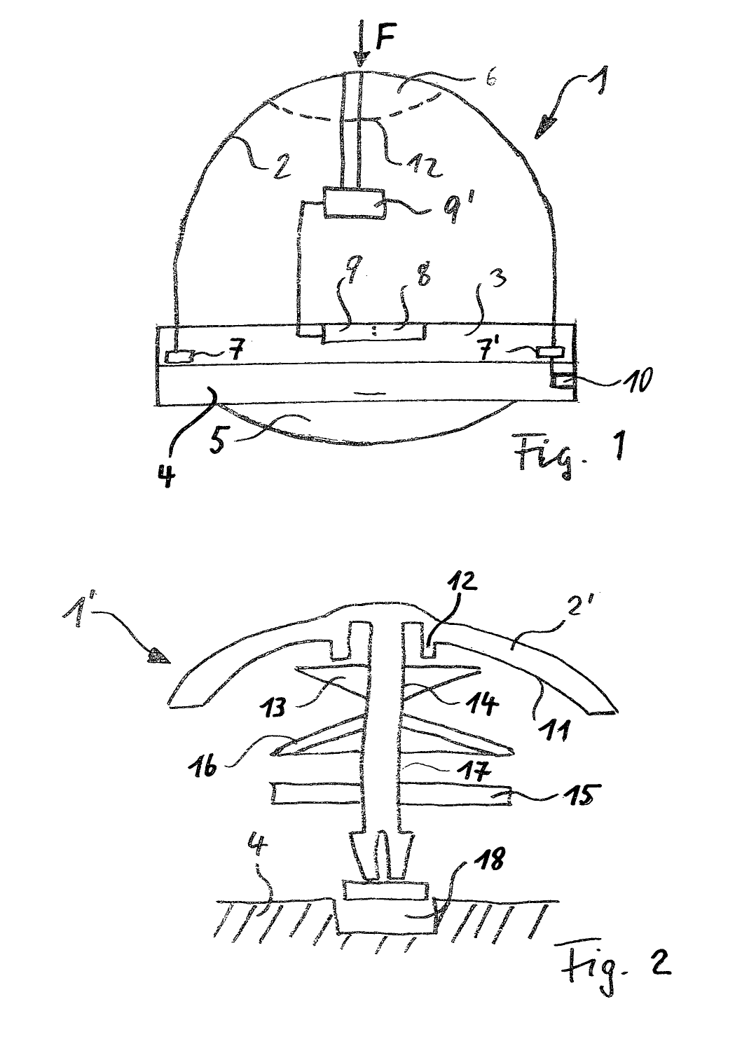

FIG. 1

is a schematic side view of an embodiment of an inventive device (1) having at least a first pressure transmitting means (2) and at least one rigid pressure receiving member (3);

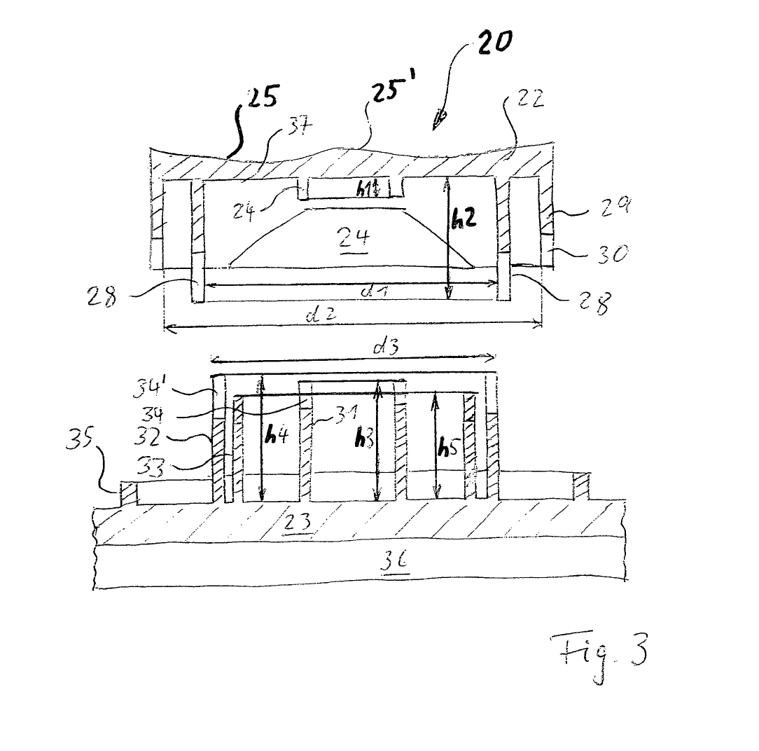

FIG. 2

a schematic exploded view of another embodiment of (1′) of the present invention, with a conical ring element (13) and a pressure receiving spring element (16) (Belleville washer);

FIG. 3

shows a schematic exploded sectional view of another embodiment (20) having a first pressure transmitting means (22) and at least one pressure-receiving member (23), and a flat one-piece spring element (24);

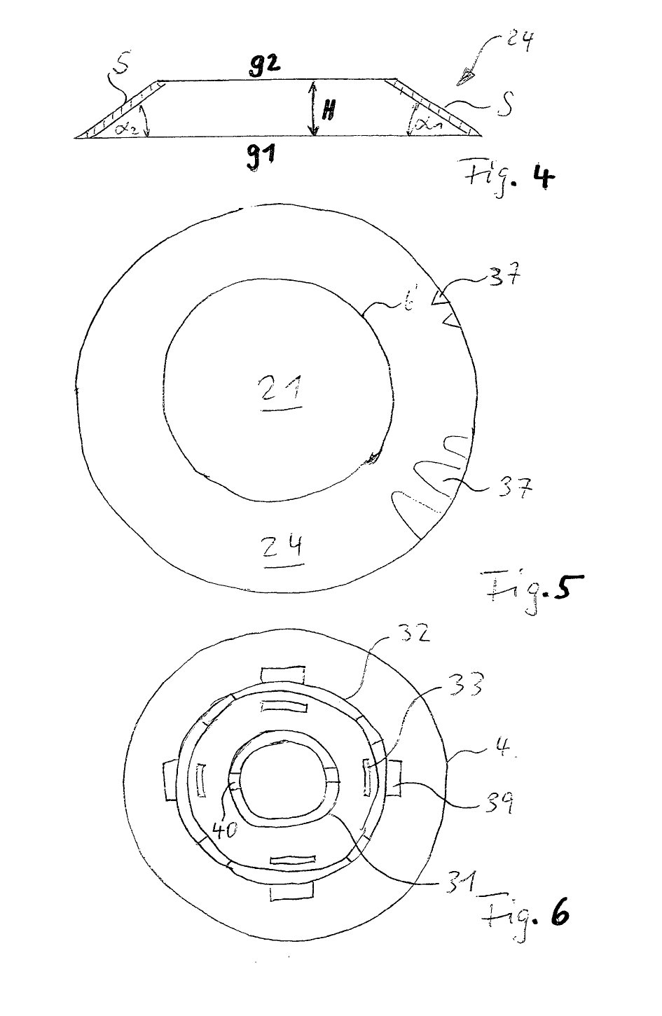

FIG. 4

a schematic cross section through a trapezoidal spring element (24) (Belleville washer);

FIG. 5

a schematic plan view of the flat ring-shaped spring element (24) with a central opening (21);

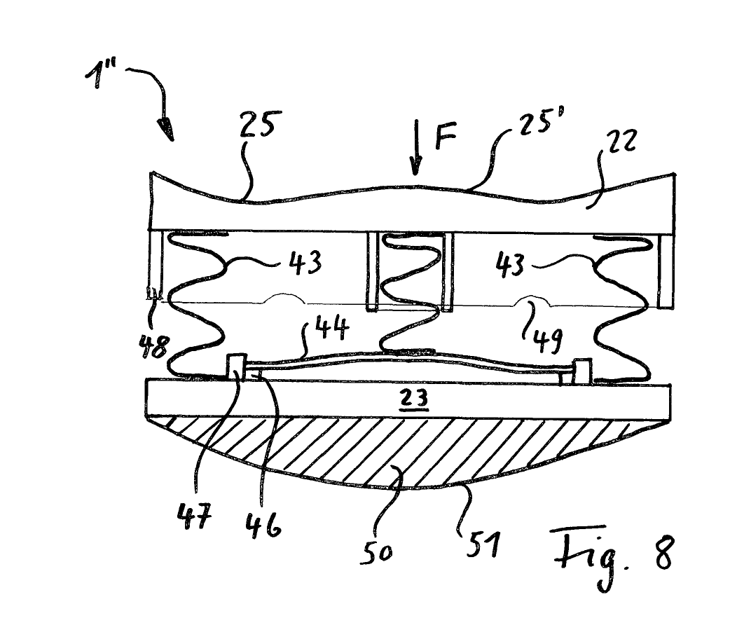

FIG. 6

a schematic plan view of the pressure-receiving rigid member (23) having concentric projections (31, 32, 33, 35), and at least one aperture (30);

FIG. 7

a schematic plan view of the pressure-rigid member (23) having concentric projections (31, 32, 33, 35) of FIG. 6, wherein a plurality are arranged spring elements 43 on a circular path around the central spring element (24, 44).

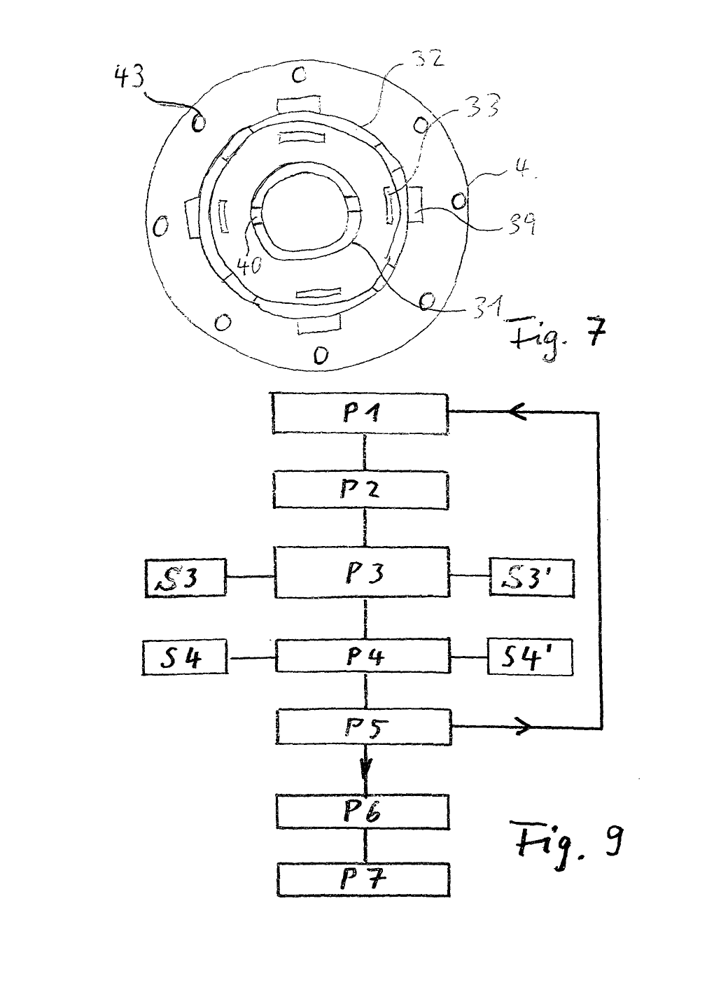

FIG. 8

is a schematic side view of an embodiment (1″) having a plurality of spring elements (43) around the central flat spring member (24, 44) around;

FIG. 9

a schematic block diagram of the individual process steps (P1-P7) during the resuscitation process.

FIG. 1 shows a schematic side view of a possible embodiment of the device 1 with its essential components. This device 1 comprises a first pressure conference agent 2, which is arc-shaped and receiving at the ends with a flat, relatively rigid pressure element 3 is connected. The rigid pressure-receiving member 3 may also be designed curved, has at its underside a layer 4 which is made of a resilient material, for example a special durable foam as foam rubber. At the bottom of the resilient layer 4, a mold part 5 is arranged that thus ensures proper and secure positioning of the device 1 on the thorax of the thorax according to the anatomical features of the thorax. The resilient member 4 and the mold part 5 can also be as a single molding from a suitable foam that does not slips on the skin of the human body, may be formed. The curved pressure transmitting means 2 is designed such that upon exercise of mechanical pressure (F) the surface of the pressure transmitting means 2 first undergoes a mechanical stress and the occurrence of the limit pressure (Fmax) thereby generate a portion of the surface abruptly buckles and a clearly perceptible clicking sound, the user indicates that the maximum pressure (Fmax) is reached. Here, the arched trained transmission means 2 serves inter alia as a resonance chamber, which increases the acoustic signal (S3) significantly. The surface 6 bends it by leaps and bounds at the predetermined pressure load (Fmax) inwardly to the dotted line 12 or similar, and thus produces the clearly audible click signal (S3). In a further development of this embodiment of the present invention are at the end of the pressure transmitting means two pressure-receiving sensors 7, 7′, for example, Strain gauges, arranged for supplying a signal of the electronics 8 and thereby a significantly perceptible acoustic or optical signal (S3′) is generated. The integrated, for example, in the pressure transmitting means 3 electronics is powered by a likewise internal or external energy storage device 9, for example, the is a battery or a capacitor. The electrical contact 10 is, inter alia, connected to the sensors 7, 7′ and can be configured as a USB socket in order to transmit more important cardiological signals.

In a further embodiment, communicates with the pressure transmitting means 2, 6, a generator 9′ (z. B. Dynamo) for energy mechanically in connection which is set by the cyclic pressure movements of the surface 2 in movement, and thereby generates power, the electric energy storage 9 is supplied.

In another embodiment, the rigid thrust receiving member 3 and the first power transmission means 2 is round, but can also be oval, but this is merely a symbolic hint, so that any other arbitrary meaningful shaping of the individual components come into question. It is important that the form is the ergonomic shapes of the hand and chest corps adapted so that all the pressure transmitting elements optimum pressurization and uniform pressure transmission initially cause the pressure transmission element 2, 3 on the one hand and on the chest corps on the other hand. The flat rigid force transmission element 3 is slightly larger than the diameter of the power transmission element 2, whereby a small edge is formed in the present embodiment. On that edge, or at another suitable location of the device 1 two marking elements are arranged which serve as positioning aids for the correct placement of the device 1 onto the chest of the human body. A further positioning aid, a flexible element, for example a ribbon or string to be, which is placed around the patient's neck, thereby quickly find the right heart position.

FIG. 2 schematically shows a further exemplary embodiment 1′ of the present invention in an exploded view of the main structural components. Here, the convex pressure transmitting means 2′ at the bottom 11 to an annular ridge 12 which acts on a conical ring element 13 with a central bore 14 with pressurizing (F). Between a further flat annular disc 15 at least one elastic thrust receiving spring element 16 is arranged, which is formed in the present embodiment, as raised plate spring made of spring steel, which at a predetermined load (Fmax) turns, that is, against the conical annular element 13 strikes. Conical disc springs made of DIN-compliant spring steel in the same direction and/or exchange intimate arrangement are suitable because of their small spring travel at high spring force especially well as a pressure receiving member 16 in the present invention. The impact of the spring element thus transmits over the annular ridge 12 on the first pressure transmission element 2′, which serves as a resonance chamber and 16 generates an easily audible sound at impact of the disk spring. The in this view looking down guide rod 17 on which radiate, not shown here, the ribs are arranged, serves among other centric and homogeneous power transfer to the molded part 4, or 5, the center has a recess 18 into which the guide rod 17 when force is applied to the first pressure transmitting means 2′ is immersed. The disc spring, or the spring pressure receiving member 16 can assume any outer form. The outer shape of the spring element 16 can be round, oval, polygonal or star-shaped molded. It is important that they at a predetermined load (Fmax) a signal of any kind, such as where described, is generated.

FIG. 3 shows a schematic sectional view of another embodiment 20 with a first pressure transmission medium 22 and at least one pressure-receiving member 23, and a spring member 24 which is disposed in principle between the first pressure transmitting means 22 and the pressure receiving member 23. The first pressure transmission medium 22 is formed in approximately U-shaped and has on its upper side a top surface 25 which is formed concave and the center may have a small increase 25′ or buckle, the ergonomic and centering purposes is used. The first pressure transmission medium 22 is formed in the present embodiment round, but this is not mandatory. Likewise, oval, heart-shaped or polygonal embodiments are conceivable. On the inside 26 of the pressure transmitting means 22, a concentric annular rib 27 is arranged, whose diameter corresponds approximately to the diameter G2 of the opening of the spring element 24. This annular rib 27 is comparatively within the first pressure transmitting means 22 small in height H1 compared to height h2 another annular prominence 28th The diameter of the projections 27 and 28 assume approximately the size of the diameter g1 and g2 of the spring element 24 and must be so large that the annular boss 28 fits over another, to be described below annular elevation 32nd The side walls 29 are also annular in shape and have a plurality of recesses 30 on. The spring element 24 is described in more detail below. Below the first pressure transmission medium 22 is a relatively rigid pressure receiving member 23 is arranged, which can be considered as a base plate on which a plurality of annular projections 31, 32, 33 are formed. The central annular projection 31 is larger than the height h1 of the annular opposite elevation 27 in the first pressure transmission medium 22, which is related to the geometry of the spring element 24. In height h3 Concentric with the annular ridge 31, a further annular projection 32 is arranged, which is h4 slightly higher in height than the central annular projection 31. The diameter of the annular ridge 31 corresponds approximately to the diameter G2 of the annular ridge 27 within the first pressure transmission medium 22 and serves, inter alia, as a guide means. In the immediate vicinity of the annular projection 32 are isolated more spring support members 33 arranged on the spring element 24 is loosely placed. The level h5 this spring support members 33 is slightly lower than the height h3 of the annular elevation 31, so that the impact of the spring element 24 when reaching the limit pressure (Fmax) vigorously strikes the middle projection 31 and upon release of the mechanical pressure (F), the spring element 24 immediately again returns to the starting position. The annular projections 31, 32, 35 each have at least one recess 34, 34′, which are distributed over the entire circumference of the projections. Concentric to the two surveys 31, 32 is another annular ridge 35 arranged whose diameter is d2 smaller than the inner diameter d1 of the first pressure transmitting means 22 below the base plate 23 is an elastic material arranged as a pressure transmission means 36 of to directly to the chest objective body is in contact. The material is a special foam, e.g. as a foam rubber, as described above.

FIG. 4 shows a schematic cross section of a trapezoidal shape formed spring member 24, the receiving between the first pressure transmitting means 22 and the pressure member 23 (base plate) is arranged. The spring element 24 is an annular disk formed in the present embodiment, approximately, with the flat sides (S) of the trapezium can be slightly curved and the angle (α1) and (α2), which are enclosed by the base line g1, and the sides (S), not must be necessarily equal. The baseline g2 practically represents the opening of the annular disc. The height H of the trapezoid plate disc is in the present embodiment, between 1.3 mm and 2.9 mm, wherein the wall thickness of the spring element 24 is approximately 0.3 mm and the outer diameter (D) is between 44 mm and 55 mm.

FIG. 5 shows the top view of the annular spring element 24 of FIG. 4. The internal diameter of the annular disc is between 7 mm and 32 mm. On the circumference of the outer diameter (g1) a plurality of deformations 37 is arranged, which can have different forms, for example, wave-shaped and/or U-shaped. Further recesses both of which can be arranged in the peripheral region 38, including acting on the stiffness of the spring element 24. Other deformations, such as e.g. Holes, are also possible for this purpose.

The FIG. 6 shows a detail of a schematic plan view of the pressure receiving portion 23 (base plate) of the device 1′. As already described, the base plate receives 23 concentrically disposed elevations, which have the circular elevations of different heights h1, h2, h3, h4, h5. The base plate 23 itself can be a flat or curved surface having at least one recess, wherein the possible openings 39 are disposed generally diametrically opposite one another, and serve, among others for engagement by a fastening hook which is arranged on the elevation 28 inside the first pressure transmission medium 22, which is pushed over the annular projection 32 on the base plate 23. The annular ridge 31 has a plurality of recesses 34, distributed over the entire circumference, on. Within the annular projection 32, in the vicinity of the openings 39 are isolated from the annular projection 32 of support members 33 for supporting the spring element 24 is arranged diametrically to each other, having a height h5. The middle annular elevation 31 in the present embodiment has a relatively narrow indentations 40, which lie opposite each other. The outer annular elevation 41 serves the delimitation of the base plate 23 and for receiving the side walls 29 of the first pressure transmission medium 22, whose diameter is approximately between 70 mm and 120 mm. The entire base plate 23 is more oval or teardrop-shaped with a longitudinal dimension of about 120 mm.

FIG. 7 shows a schematic plan view of the pressure-receiving rigid member 23 with concentric elevations (31, 32, 33, 35) of FIG. 6, wherein a plurality of spring elements 43 are arranged concentrically on a circular path and with the upper pressure transmitting means 2, 22 is in communication in order to concentrate the mechanical pressure (F) exerted on the first pressure transmission means and to transmit at least one flat spring element 24 to the. Thus, the first pressure transmitting means 22 is practically a floating cushion.

FIG. 8 shows a schematic side view of a further embodiment of the inventive device 1″ to the cardiopulmonary resuscitation of a human body with a plurality of spring elements 43 to the center spring member 44 around, above which a further spring element 45 is arranged, the with its one end, the surface of the flat spring member 44 contacts. The spring elements 43 are female between the first pressure transmitting means 2, 22 and the pressure element 3 is arranged, wherein the one end of the spring element 43, the underside of the first pressure conference agent 2, 22 contacts and the other end of the inside of the pressure receiving member 23. The at least two Spring elements 43 are formed in the present embodiment as a helical pressure springs which transmit the mechanical pressure (F) from the first pressure transmission medium 22 to the pressure receiving member 23. Simultaneously, the pressure of the first pressure transmitting means 2′ by means of the other spring element 45 is transmitted to the sheet-like spring element 44, whereby the sheet-like spring element is deflected 44 when force is applied to the first pressure transmission medium 22 downward and spontaneously jumps at a predetermined deflection in a final deflection and thereby a clearly perceptible signal (S3) is generated. Any decrease of mechanical pressure (Fmax) to the first pressure transmitting means 22 the flat spring member 44 jumps back to the starting position and proposes it to the surface at the lower end of the spring element 45, whereby a second clearly discernable signal (S4) is generated. The further spring element 45 is seated in a guide 46, which simultaneously serves as a resonance chamber in cooperation with the spring elements 44, 45. The spring constant of the spring elements 43 (coil springs) are chosen such that the first perceptible signal (S3) is generated at an adjustable mechanical force between 40 kg and 50 kg and the second visual signal (S4) falls below the set force (F), acting on the force transmitting means 22. The adjustment of the mechanical force (Fmax) is carried out by the interaction of all spring elements involved. The sheet spring member 44 is exposed to on a plurality of supporting elements 46, the edge of the flat spring member 44 is supported by lateral supports 47. The upward-facing surface 25 of the first power transmission means 22 is formed substantially concave, whereby for certain applications, the surface centrally a slight agglomeration 25′ has. The special design of the bowl-shaped surface is a decentralized power exercised during treatment of patients is important. The spring elements 43 (coil springs) are surrounded by a lateral boundary 48 which has a plurality of (arcuate) recesses 49 at the bottom. Underneath the force receiving element 23 is at least one layer 50 of a resilient flexible material, eg. B foam, arranged whose outwardly facing surface 51 is substantially convex to favorably adapt to the anatomy of the chest carcass.

FIG. 9 shows a block diagram of the essential process steps (P1-P7) upon application of the inventive device 1. The first step P1 is effective to set up the device on the rib cage at a predetermined location and position. The second step P2 is to hand a mechanical pressure (F) on the surface 24, 25 of the pressure transmitting means 2, 2′ 22 of the device 1, 1′, 1″ to exert 20 until a threshold pressure (Fmax) reaches, wherein the threshold pressure is about between 40 and 50 kg, but preferably is applied at 45 kg. After reaching the limiting pressure (Fmax) is determined by the device 1 from a human sense organ perceivable signal (S3, S3′), generated either mechanically and/or electrically, the users says that the limit pressure (Fmax) is reached. Once this limit pressure has been reached, the mechanical pressure immediately completely withdraw (P4), thereby producing a second signal (S4, S4′) is created. After a short predetermined time in a further process step (P5) is then to repeat the printing operation cyclically.

In an embodiment of the present invention to provide an integrated electronic circuit 9 is as described above, is provided which serves to produce a current-voltage pulse, so that after the heart-lung massage (P1-P5) comprises the steps P6—follow P7, wherein P6 means the application of the electrodes of the defibrillator and P7 the discharge by means of a specific signal of the respective energy accumulator.

The features of the embodiments described above can of course be combined with each other so that a characteristic of the one embodiment may be included in another embodiment without departing from the scope of the basic inventive concept.

1. A device (1) for the controlled cardiopulmonary resuscitation of the human body in cardiac arrest, with at least one pressure transmitting means (2, 22) and at least one pressure-receiving member (3, 23) and a pressure indicator, which upon the occurrence of an adjustable limit pressure (Fmax) generates a perceivable by human sense organs signal (S), characterized by at least one spring system with a plurality of spring elements (6, 16, 24, 43, 44, 45) which cooperate when exercising a mechanical pressure (F), wherein at least one spring element (6, 16, 24, 44) is flat and one piece and at least two spring elements (43) laterally to the sheet-like spring element (6, 16, 24, 44) are arranged.

2. Device according to claim 1, characterized in that on reaching the adjustable threshold pressure (Fmax) and the jump back to the start position of a clearly audible signal (S) is generated, which both mechanically and acoustically to the spring element (4) carrying elements acts.

3. A device according to claim 1, characterized in that the cross-sectional shape may be trapezoidal through the center of the spring element (24), wherein G1 represents the lower baseline, and g2, the upper baseline of the trapezoid.

4. The apparatus of claim 1, characterized in that the two sides are taking s1 and s2 of the trapezoid with the base line g1 an acute angle (α1, α2), where (α1) and (α2) are not necessarily equal.

5. A device according to claim 1, characterized in that the wall thickness (d) of the planar one-piece casing of the trapezium is of between 0.5 and 1 mm, preferably 0.3 mm, at a suitable spring steel.

6. The device according to claim 1, characterized in that the flat spring member (24, 44) may be formed of an annular disk.

7. The device according to claim 1, characterized in that the edge regions of the inner and outer periphery of the disk spring (24, 44), wave-shaped and/or polygonal deformations (37) and recesses (38) and/or bores, which influence the stiffness of the spring element and define.

8. A device according to claim 1, characterized in that the lateral surfaces, or sides s1 and s2 of the trapezoidal cross-section spring element (24, 44) can be easily formed spherically.

9. Device according to claim 1, characterized in that the outer diameter (g1) of the spring element (24, 44) between about 30 mm and about 75 mm, preferably 47 mm and the inner diameter (g2), if provided, between 9 mm and 20 mm varies, preferably may be at 16 mm.

10. The device (1) for the controlled cardiopulmonary resuscitation of the human body in cardiac arrest, characterized in that on a flat or curved base surface (23) with at least one aperture (39) concentric elevations (32, 33, 34, 35 are arranged) receiving the centrally least one spring element (24, 44), wherein the elevations circular, polygonal or wave-shaped as rings with at least one recess (34, 34′) may be arranged.

11. The device according to claim 10, characterized in that at least one spring support element (33) isolated from its neighboring projection (32) is arranged.

12. Device according to claim 10, characterized in that on the outside of the base surface (23), a foam (36) is arranged whose surface is formed spherically.

13. A device according to claim 10, characterized in that the spring element (24, 44) is arranged between the base plate (23) and at least one pressure-receiving member (2, 22), wherein the pressure-receiving member (2, 22) on its inner side having at least one concentric collection, which has at least a circular elevation with at least one recess and the at least one elevation arranged in a circle about the diameter (G2) of the opening (21) of the spring element (4, 24).

14. Device according to claim 1, characterized in that the top surface (25) of the cylinder-shaped at least one pressure transmitting means (22) is concave, the upper surface may have at least a curvature (25′) and the side wall (29) at least one recess (30), preferably a plurality of recesses.

15. Device according to claim 10 characterized in that the at least one spring element (24, 44) is formed back expanding while at least a second signal (S4) generated.

16. A device according to claim 1, characterized in that, that is disposed around the at least one two-dimensional one-piece spring element (24, 44), at least one further spring element (43), the mechanical pressure on the at least one pressure transmitting means (2, 22) receiving and indirectly on the flat spring member (44) transfers.

17. A device according to claim 1, characterized in that the further spring elements (43) to the first flat spring member (24, 44) can be arranged, which are formed in one embodiment as spiral springs.

18. A device according to claim 1, characterized in that, that above the sheet-like spring element (24, 44) at least one spring element (45) is arranged.

19. Device according to claim 1, characterized by an integrated electronic system (8) with at least one sensor (7) which accommodates the mechanical pressure (F) and the electronics (8) a signal (S) generated.

20. Device according to claim 1, characterized in that the electronics (8) from an internal or external power source (9) is supplied.

21. Device according to claim 1 characterized in that at a suitable position at least one electrical contact is arranged, the receiving at least one pressure element (3, 23) is in communication.

22. Device according to claim 1, characterized in that an electronic unit (9′) (Defibrillator) which outputs based on a signal (S) an adjustable current pulse which acts on the body to be treated of the patient.
